(19) United States
(12) Patent Application Publication
(10) Pub. No.: US 2017/0239169 A1
(43) Pub. Date: Aug. 24, 2017

(54) Illuminating and Pore Refining Complexes

(71) Applicant: Marula Cosmetics Inc. dba Yangu Beauty, Southlake, TX (US)

(72) Inventors: Sipho Gumbo, Southlake, TX (US); Joy E. Roederer, Richardson, TX (US)

(73) Assignee: Marula Cosmetics Inc. dba Yangu Beauty, Southlake, TX (US)

(21) Appl. No.: 15/438,728

(22) Filed: Feb. 21, 2017

Related U.S. Application Data
(60) Provisional application No. 62/297,268, filed on Feb. 19, 2016.

(51) Int. Cl. A61K 8/97; A61K 8/19; A61K 8/14; A61K 8/92; A61K 8/06

(52) U.S. Cl. CPC A61K 8/97; A61K 8/922; A61K 8/062; A61K 8/14; A61K 8/19

(57) ABSTRACT

An illuminating complex for enhancing beauty includes at least one of yangu seed oil, baobab seed oil, mafura butter (trichilia oil), dipotassium glycyrrhizinate, alpha-arbutin, and a structure forming agent. A pore refining complex includes at least one of kalahari melon seed oil, grapeseed oil, rooibos tea, an extract blend of at least one of witch hazel, white willow bark, acacia senegal, papaya seed, and lemongrass, and a structure forming agent. The structure forming agent preferably comprises at least one of an emulsification, an encapsulation, and liposomes.

### CROSS-REFERENCE TO RELATED APPLICATIONS

This application claims the benefit of U.S. Provisional Application No. 62/297,268, filed Feb. 19, 2016, which application is hereby incorporated herein by reference, in its entirety.

### TECHNICAL FIELD

The invention relates generally to compositions and methods for enhancing a person's beauty and, more particularly, to compositions and methods for enhancing a person's beauty using illuminating and pore refining complexes.

### BACKGROUND

Current state of the art in skin care is that products are made to work for the generalized majority of skin types, but leave African consumers without skin care products that address their specific needs.

By way of example, skin lightening products generally produce an overall, usually aggressive process for lightening of the skin. Current products on the market do not address the specific pigmentation issues of African skin, which has unique characteristics and needs. For example, certain stressors (scarring, acne, inflammatory responses) will cause African skin to develop localized hyperpigmentation and this process is not true for other skin types. Likewise, treatment of the localized pigmentation is a more delicate process for African skin so that it does not actually cause more damage and resulting in worsening of the hyperpigmentation.

State of the art consists, usually, of using astringents to dry up sebum and minimize pore size. Other products on the market use natural plant extracts that are known to act as astringents. However, this treatment effectively dries out the skin. One usually follows such treatment with a moisturizer to counteract the effects of the astringent. This treatment regimen will not work for African skin, which displays both overproduction of sebum and dry skin at the same time, so that using a strong astringent will cause overdrying and damage to the skin.

In view of the foregoing, it is clear that there is a need for skin care products that address the specific needs of African consumers.

### SUMMARY

Accordingly, in one embodiment of the present invention, a system is disclosed which reduces the pigmentation of dark spots on African skin, while maintaining the skin's integrity and healthy state to prevent further damage. This embodiment provides an illuminating complex for enhancing beauty, the complex preferably including at least one of yangu seed oil, baobab seed oil, mafura butter (trichilia oil), dipotassium glycyrrhizinate, alpha-arbutin, and a structure forming agent. The complex of this embodiment is referred to herein as the “YMB” complex, for the three main ingredients, yangu seed oil, mafura butter (trichilia oil), and baobab seed oil. Alternatively, “YMB” may be an abbreviation for “Yangu My Beauty”.

In a further embodiment of the present invention, a system is disclosed which uses plant-derived active ingredients that control sebum production in a way that works with the skin's own processes, combined with ingredients that soothe and calm the skin and prevent loss of moisture. More particularly, this embodiment comprises a pore refining complex which preferably includes at least one of kalahari melon seed oil, grapeseed oil, rooibos tea, an extract blend of at least one of witch hazel, white willow bark, acacia senegal, papaya seed, and lemongrass, and a structure forming agent.

A structure forming agent for either of the above embodiments preferably includes at least one of an emulsification, an encapsulation, and liposomes.

A delivery vehicle for either embodiment is designed to use skin-identical lipid technology to protect and transport the active ingredients into the skin where they can be most effective.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### DETAILED DESCRIPTION

The following description is presented to enable any person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the spirit and scope of the present invention. Thus, the present invention is not intended to be limited to the embodiments shown, but is to be accorded the widest scope consistent with the principles and features disclosed herein. Additionally, as used herein, the term “substantially” is to be construed as a term of approximation.

The present invention teaches at least two embodiments for enhancing one's beauty, namely, (1) an illuminating complex, and (2) a pore refining complex. These are described in further detail below.

The illuminating complex, also referred to as YMB™, is a skin-nourishing complex comprising ingredients preferably sourced from Africa, though they could be sourced from one or more other countries or regions, and which ingredients are known for their benefits for beautiful skin. Rich hydrating oils combine with natural pigment-correcting ingredients to give the skin a more even tone and beautiful glow. These ingredients are formulated into a complex that helps protect and deliver the active ingredients to the skin. The ingredients preferably include yangu seed oil, baobab seed oil, mafura butter, dipotassium glycyrrhizinate, and alpha-arbutin, discussed in further detail below.

Yangu Seed Oil (Calodendrum Capense) is formed from oil which is cold pressed from the ripe seeds of the Cape Chestnut tree. Yangu seed oil contains skin-identical essential fatty acids that nourish skin and reinforce protective lipid layer, and exhibits natural UV protection and antioxidant properties.

Baobab Seed Oil (Adansonia Digitata) is formed from oil which is cold pressed from the seeds of the Adansonia tree. Baobab seed oil absorbs quickly without oily residue, and is a natural source of vitamins A and F, and omega fatty acids 6 and 9. Traditionally, baobab seed oil has been used for moisturizing, skin tone, elasticity, and skin healing.

Mafura Butter (Trichilia Oil) (Trichilia Emetica Seed Butter) is cold pressed from the entire seed of the Cape Mahogany tree to obtain both the oil and solid “butter” portions together (these are obtained from different parts of the seed and can be pressed separately, though the entire seed may be used). The butter forms at room temperature, and melts at “body temperature” (86° F.). Mafura butter is rich in essential fatty acids, and high in oleic acid which protects lipids from oxidation, both in this invention and in the skin's lipid bilayer. Traditionally, mafura butter has been used for healing, antimicrobial, and anti-inflammatory properties (contains Trichilin A).

Dipotassium Glycyrrhizinate (Dipotassium Glycyrrhizate) is obtained by water-extraction from licorice root, and contains saponins, flavonoids, chalcones, isoflavones, coumarins, stilbenoids, asparagines, and polysaccharides. Dipotassium glycyrrhizinate is effective for natural skin-lightening with gentle, non-irritating action, and it exhibits anti-inflammatory, antioxidant, anti-irritant, and antimicrobial functions.

Alpha-Arbutin (Arbutin) is extracted from bearberry fruit, and is a derivative of hydroquinone, but safer, as it is not as sensitizing, irritating, or toxic. Alpha-Arbutin inhibits tyrosinase to prevent the formation of melanin, and can aid in degradation of melanin over time. Alpha-Arbutin provides effective treatment for hyperpigmentation caused by a variety of triggers including inflammation and scarring, age spots (lentigines), uneven pigmentation, melasma, chloasma, and freckles.

The active components described above are preferably formulated into a complex using a structure-forming agent selected from at least one of an emulsification, an encapsulation, and liposomes, described below.

There are two types of emulsification, namely, an oil-in-water (O/W) emulsifier and a water-in-oil (W/O) emulsifier. An O/W emulsifier is preferred for embodiments comprising water or other polar or hydrophilic carrier (such as glycerin) as the primary phase. Examples of an O/W emulsifier include carbomers, polyacrylates, polyacrylamides, polysorbates, and other ingredients or blended ingredients used as O/W emulsifying agents. A W/O emulsifier is preferred for embodiments comprising oil as the primary phase. Examples of a W/O emulsifier include stearates, lactylates, oleates, alcohols, and other ingredients or blended ingredients used as W/O emulsifying agents.

Encapsulation uses physical entrapment to hold the active ingredients and release them over time or on a trigger such as contact with skin lipids, enzymes, or moisture. Encapsulation can be in the form of liquid, powder, or bead. Encapsulation material preferably comprises waxes, polyvinyl alcohol, alcohols, butters, starches, and other ingredients used to entrap active molecules.

Liposomes use lipid bilayers to incorporate, protect, and deliver the active ingredients to the skin. Liposomes preferably comprise lecithin, phosphatidylcholine, and other liposome-forming phospholipids.

In one preferred embodiment, an oil-in-water (O/W) emulsion having the consistency of a thick cream or gel, comprises the following:

| Range (approx.) | Ingredient | Preferred (example) |
|---|---|---|
| 0-95% | Yangu Seed Oil | 27% |
| 0-95% | Baobab Seed Oil | 27% |
| 0-95% | Mafura Butter | 27% |
| 0-50% | Dipotassium Glycyrrhetinate | 2% |
| 0-50% | Arbutin | 2% |
| 0-95% | Glycerin | 9% |
| 0-95% | Water | 0 |
| 0-50% | Emulsifier | 5% of W/O emulsifier |
| 0-10% | Preservative | 1% |

It is noted that the above emulsifier can be chosen from any O/W or W/O system. A preferred emulsifier will be cold-processed, such as a blend of: caprylic/capric triglycerides, glycerin, water, sucrose laurate, and/or sodium stearoyl lactylate.

In accordance with principles of one preferred embodiment of the present invention, (1) DPG and arbutin are pre-mixed in the glycerin. (2) In a separate (main) vessel, the W/O emulsifier is added and mixing begins on high speed. Begin adding the oils slowly with mixing. (3) The first 10% of the oil phase is added and mixed until substantially blended, about 5 minutes. (4) An additional 40% of the oil phase is slowly added to the mixing vessel, over the next approximately 5 minutes. (5) The remaining oils (last 50%) are slowly added over the next approximately 10 minutes. The mixing speed is increased as the gel thickens and mixing continues until substantially homogeneous. (6) The glycerin phase is added to the main vessel and mixed until all components are substantially emulsified and the mixture is substantially uniform.

There are a number of advantages associated with using YMB. For example, it provides (1) a unique combination of active ingredients that work together for evening skin pigmentation and improving skin health. (2) There are many possible levels of each constituent, ranging from 1-99% of the complex, with one preferred formulation given as an example. (3) Oils are selected for high oleic acid content to provide protection against oxidation for all components in the finished complex. (4) YMB is formulated into a complex that protects the oils and plant extracts from oxidation and premature reaction until they are delivered to the skin. (5) Composition of the complex includes but is not limited to emulsion (O/W or W/O), micelle, and liposome. (6) A delivery vehicle includes but is not limited to (a) cosmetic cream, lotion, gel or liquid, (b) powder (dehydrated, freeze-dried, or spray-dried), (c) gel, and (d) stick or roll-on.

In a second embodiment of the invention, a skin-clarifying complex referred to herein as Yangu Beauty Pore Refining Complex™, contains ingredients preferably sourced from Africa, though they could be sourced from one or more other countries or regions, and which ingredients are known for their benefits for clear, beautiful skin. This is a vitamin-enriched complex which helps reduce the appearance of pores and gives the skin a more clear, radiant complexion. These ingredients are formulated into a complex that helps protect and deliver the active ingredients to the skin. Ingredients preferably include kalahari melon seed oil, grapeseed oil, rooibos tea, and an extract blend of at least one of witch hazel, white willow bark, acacia senegal, papaya seed, and lemongrass.

Kalahari Melon Seed Oil (Citrullus Vulgaris) is formed from cold pressed seeds of preferably African watermelon, though watermelon could be from one or more other countries or regions. Kalahari Melon Seed Oil is rich in omega 6 and 9 essential fatty acids, and balances moisture and dissolves sebum build-up, helping to keep pores clear and refined. Moreover, it has a non-greasy feel.

Grapeseed Oil (Vitis Vinifera) is formed from cold pressed seeds of white grapes. Grapeseed Oil is rich in essential fatty acids, Vitamin E, and polyphenols. Furthermore, it exhibits anti-inflammatory, antioxidant, anti-histamine properties which make it beneficial for skin that is dry, irritated, or acne-prone.

Rooibos Tea (Aspalathus Linearis) is extracted from leaves of preferably South African red bush (Rooibos plant). Rooibos Tea is rich in minerals and flavonoid antioxidants, and has anti-inflammatory properties which help calm the skin. Rooibos Tea also has a high content of ascorbic acid (Vitamin C) and polyphenols, and contains Zinc which is known to help minimize sebum production to help control acne and eczema.

An extract blend comprises at least one of witch hazel, white willow bark, acacia senegal, papaya seed, and lemongrass. Witch Hazel (Hamamelis Virginiana) is extracted from leaves and bark of the preferably North American witch-hazel shrub, and is known for astringent and anti-inflammatory properties, helping to maintain clear complexions and minimize appearance of pores. White Willow Bark (Salix Alba) is extracted from the bark of the white willow tree, is anti-inflammatory, helps maintain clear complexions and reduce redness, and contains salicin, known for pain-relief properties. Acacia senegal (Acacia Senegal) is extracted from the fruit of the thorntree preferably in the Sudan in Africa, and exhibits a calming property that helps reduce redness and skin irritations, and further exhibits antimicrobial and astringent properties to relieve acne symptoms. Papaya Seed (Carica Papaya) is extracted from the seeds of the papaya fruit, either as an extract or an oil, contains the enzyme Papain which provides many benefits including exfoliation and smoothing of skin, as well as recovery from lesions such as acne and other wounds, and exhibits wound healing properties of papaya, and further contains carotenoids, flavonoids, and Vitamins A and C which are antioxidants and nourishing agents for healthy skin. Lemongrass (Cymbopogon Flexuosus) is obtained by steam distillation of dried lemongrass plant, has anti-inflammatory and antimicrobial properties which help calm skin aggravations, is known to help achieve clear complexion, and exhibits an astringent property, known to help contract pores and hair follicles.

The active ingredients described above will preferably be formulated into a complex using a structuring form agent selected from emulsification, encapsulation, and liposomes, as described in greater detail below.

Emulsification may comprise an O/W emulsifier for embodiments comprised with water or other polar or hydrophilic carrier (such as glycerin) as the primary phase. Examples of O/W emulsifiers include carbomers, polyacrylates, polyacrylamides, polysorbates, and other ingredients or blended ingredients used as O/W emulsifying agents. Alternatively, emulsification may comprise a W/O emulsifier for embodiments comprising oil as the primary phase. Examples of W/O emulsifiers include stearates, lactylates, oleates, alcohols, and other ingredients or blended ingredients used as W/O emulsifying agents.

Encapsulation may use physical entrapment to hold the active ingredients and release over time or on a trigger such as contact with skin lipids, enzymes, or moisture. Encapsulation may be in the form of liquid, powder, or bead. Encapsulation material may comprise waxes, polyvinyl alcohol, alcohols, butters, starches, and/or other ingredients used to entrap active molecules.

Liposomes use lipid bilayers to incorporate, protect and deliver the active ingredients to the skin. Liposomes preferably comprise lecithin, phosphatidylcholine, and other liposome-forming phospholipids.

In one preferred embodiment, an oil-in-water (O/W) emulsion having the consistency of a thick cream or gel, preferably comprises the following:

| Range (approx.) | Ingredient | Preferred (example) |
|---|---|---|
| 0-95% | Kalahari Melon Seed Oil | 33% |
| 0-95% | Grapeseed Oil | 33% |
| 0-95% | Rooibos Tea | 10% |
| 0-95% | Witch Hazel extract | 2% |
| 0-95% | White Willow Bark extract | 2% |
| 0-95% | Acacia extract | 2% |
| 0-95% | Papaya extract | 2% |
| 0-95% | Lemongrass extract | 0 |
| 0-95% | Glycerin | 10% |
| 0-95% | Water | 0 |
| 0-50% | Emulsifier | 5% of W/O emulsifier |
| 0-10% | Preservative | 1% |

It is noted that emulsifiers can be chosen from any O/W or W/O system. A preferred emulsifier will be cold-processed such as a blend of: caprylic/capric triglycerides, glycerin, water, sucrose laurate, and/or sodium stearoyl lactylate.

In accordance with principles of one preferred embodiment of the present invention, (1) water-soluble extracts are pre-mixed in the glycerin. (2) In a separate (main) vessel, the W/O emulsifier is added and mixing begins on high speed. Begin adding the oils slowly with mixing. (3) The first 10% of the oil phase is added and the ingredients are mixed until substantially blended, about 5 minutes. (4) An additional 40% of the oil phase is slowly added to the mixing vessel, over the next approximately 5 minutes. (5) The remaining oils (last 50%) are slowly added over the next approximately 10 minutes. The mixing speed is increased as the gel thickens and mixing continues until substantially homogeneous. (6) The glycerin phase is added to the main vessel and the ingredients are mixed until all ingredients are substantially emulsified and the mixture is substantially uniform.

There are a number of advantages associated with using the Yangu Beauty Pore Refining Complex™, including (1) the unique combination of active ingredients that work together for reducing the appearance of pore size and acne lesions. (2) There are numerous possible levels of each constituent ranging 1-99% of the complex, with one preferred formulation given as an example. (3) Oils are selected for high oleic acid content to provide protection against oxidation for all components in the finished complex. (4) The ingredients are formulated into a complex that protects the oils and plant extracts from oxidation and premature reaction until they are delivered to the skin. (5) The composition of the complex includes, but is not limited to, (a) emulsion (either 01W or W/O), (b) micelle, or (c) liposome. (6) A delivery vehicle includes, but is not limited to, (a) cosmetic cream, lotion, gel, or liquid, (b) powder (e.g., dehydrated, freeze-dried, or spray-dried), (c) a gel, or (d) a stick or roll-on.

At least one type of the aforementioned liposomes are described in further detail in U.S. Pat. No. 7,282,224, issued Oct. 16, 2007, and incorporated herein by reference in its entirety.

Having thus described the present invention by reference to certain of its preferred embodiments, it is noted that the embodiments disclosed are illustrative rather than limiting in nature and that a wide range of variations, modifications, changes, and substitutions are contemplated in the foregoing disclosure and, in some instances, some features of the present invention may be employed without a corresponding use of the other features. Many such variations and modifications may be considered obvious and desirable by those skilled in the art based upon a review of the foregoing description of preferred embodiments. Accordingly, it is appropriate that the appended claims be construed broadly and in a manner consistent with the scope of the invention.

1. An illuminating complex for enhancing beauty, the complex comprising at least one of:
yangu seed oil;
baobab seed oil;
mafura butter (trichilia oil);
dipotassium glycyrrhizinate;
alpha-arbutin; and
a structure forming agent.

2. The illuminating complex of claim 1, further comprising at least one of glycerin, water, emulsifier, and a preservative.

3. The illuminating complex of claim 1, wherein the structure forming agent comprises an emulsification.

4. The illuminating complex of claim 1, wherein the structure forming agent comprises an encapsulation.

5. The illuminating complex of claim 1, wherein the structure forming agent comprises an liposomes.

6. The illuminating complex of claim 1, further comprising a delivery vehicle configured for applying the complex to skin.

7. The illuminating complex of claim 1, further comprising a delivery vehicle, wherein the delivery vehicle is at least one of a cosmetic cream, lotion, gel, and liquid.

8. The illuminating complex of claim 1, further comprising a delivery vehicle, wherein the delivery vehicle is at least one of a dehydrated powder, freeze-dried powder, and spray-dried powder.

9. The illuminating complex of claim 1, further comprising a delivery vehicle, wherein the delivery vehicle is a gel.

10. The illuminating complex of claim 1, further comprising a delivery vehicle, wherein the delivery vehicle is at least one of a stick and roll-on.

11. A pore refining complex comprising at least one of:
kalahari melon seed oil;
grapeseed oil;
rooibos tea;
an extract blend of at least one of witch hazel, white willow bark, acacia senegal, papaya seed, and lemongrass; and
a structure forming agent.

12. The pore refining complex of claim 11, further comprising at least one of glycerin, water, emulsifier, and a preservative.

13. The pore refining complex of claim 11, wherein the structure forming agent comprises an emulsification.

14. The pore refining complex of claim 11, wherein the structure forming agent comprises an encapsulation.

15. The pore refining complex of claim 11, wherein the structure forming agent comprises an liposomes.

16. The pore refining complex of claim 11, further comprising a delivery vehicle configured for applying the complex to skin.

17. The pore refining complex of claim 11, further comprising a delivery vehicle, wherein the delivery vehicle is at least one of a cosmetic cream, lotion, gel, and liquid.

18. The pore refining complex of claim 11, further comprising a delivery vehicle, wherein the delivery vehicle is at least one of a dehydrated powder, freeze-dried powder, and spray-dried powder.

19. The pore refining complex of claim 11, further comprising a delivery vehicle, wherein the delivery vehicle is a gel.

20. The pore refining complex of claim 11, further comprising a delivery vehicle, wherein the delivery vehicle is at least one of a stick and roll-on.
